(19) United States
(12) Patent Application Publication
(10) Pub. No.: US 2023/0340558 A1
(43) Pub. Date: Oct. 26, 2023

(54) METHOD FOR ANALYZING A BIOLOGICAL SAMPLE COMPRISING AN INITIAL COMPLIANCE ANALYSIS

(71) Applicant: BIOMERIEUX, Marcy L'Etoile (FR)

(72) Inventors: Dominique DECAUX, Chaponost (FR); Emilie BISCEGLIA, La Tour de Salvagny (FR); Guillaume PERRIN, Tassin la Demi-Lune (FR)

(73) Assignee: BIOMERIEUX, Marcy L'Etoile (FR)

(21) Appl. No.: 18/026,677

(22) PCT Filed: Sep. 23, 2021
(86) PCT No.: PCT/FR2021/051634

§ 371 Date: Mar. 16, 2023

(30) Foreign Application Priority Data
Sep. 25, 2020 (FR) 2009777

(51) Int. Cl. C12Q 1/18; G01N 21/59; G03H 1/00

(52) U.S. Cl. CPC C12Q 1/18; G01N 21/59; G03H 1/0005; G03H 2001/005

(57) ABSTRACT

Systems and methods for determining a topology for a multi-path connection based on one or more application-level wherein, a method performed by a master topology function includes receiving a request for a topology for a multi-path connection that satisfies an application-level requirement(s) of a particular application and determining a topology for the multi-path connection based on the application-level requirement(s). The topology includes two or more links from a source wireless node to a target wireless communication device and at least one of the two or more links is a multi-hop link from the source wireless node to the target wireless communication device via one or more additional wireless communication devices that operate as relays. The method further includes configuring the source wireless node, the target wireless communication device, and the one or more additional wireless communication devices in accordance with the determined topology.

### TECHNICAL FIELD

The invention relates to the field of the analysis of biological samples by imaging, and more particularly relates to the checking of the compliance of a biological sample in the context of the analysis of biological agents in the biological sample.

### TECHNOLOGICAL BACKGROUND

The analysis of biological samples by imaging makes use of an optical analysis instrument into which the biological samples to be analyzed are introduced. A biological sample consists of a suspension of biological agents or of a mixture of suspension of biological agents. The biological agents are for example micro-organisms (bacteria, yeasts, mold, etc.). The analysis of the biological agent in the biological sample can comprise identifying said biological agent or determining a characteristic of this biological agent, such as for example the minimum inhibitory concentration of an antibiotic that would be effective against said biological agent.

The biological sample, known as inoculant, in its initial state is placed in an at least partially transparent receptacle, or well, through which the analysis instrument can perform optical property measurements on the biological sample. The well contains a nutritive medium and also one or more reagents, such as an enzymatic substrate or antibiotics, intended to interact with biological agents present in the biological sample. Generally, a plurality of wells are provided to receive each inoculant, each of the wells containing different reagents or one and the same reagent at different concentrations. Depending on the nature of the biological agents present in the inoculant, said agents react with certain reagents, and not with other reagents, or with certain concentrations and not with others. For example, in the context of an antibiogram for testing antibiotic sensitivities, the reagents consist of various antibiotics at various concentrations, and the biological agents will multiply in the wells containing the antibiotics to which they are not sensitive or in which the concentration of antibiotics is insufficient, or conversely, the growth of said biological agents will be more or less hindered in the wells containing the antibiotics to which they are sensitive at sufficient concentrations.

These differences in interactions between the biological agents and the reagents therefore result in different changes in the biomass in the wells. The biomass, that is to say the amount of biological material present in each well, directly influences the optical properties of the biological sample present in each well, since the biological agents themselves have optical properties that are different from the solution in which they are in suspension.

In particular, the transmittance of the biological sample is affected by the change in the concentration of biological agents. For this reason, methods had been developed for analyzing biological samples based on determining the change over time, during an incubation phase, of the overall transmittance (or absorbance, which is equivalent) of a well filled with the biological sample, in order to determine therefrom a turbidity measurement, typically expressed in McFarland (McF). This turbidity measurement is directly representative of the biomass of biological agents in the biological sample. To do this, an emitting diode illuminates the sample with a light beam of known intensity, and an isolated photodiode placed opposite the emitting diode relative to the sample makes it possible to determine the light intensity received after the light beam has passed through the biological sample. However, such a transmittance measurement has quite a low sensitivity, such that it is not possible to measure a turbidity of less than 0.05 McF, or even less than 0.1 McF.

The inoculum is prepared by an operator who introduces biological agents in suspension into a saline solution or by diluting a biological sample (positive urine or blood culture for example) so as to obtain a bacterial concentration between 10⁷ and 10⁹ UFC/ml. The dilution in the saline suspension must initially correspond to a specific range in order to allow the analysis. This compliance range can be expressed directly as turbidity value for the intention of the operator preparing the inoculant, optionally with a prior value which is later rediluted. By way of example, for some protocols, a presuspension must be calibrated between 0.5 and 0.63 McF for bacteria as biological agents or else between 1.8 and 2.2 McF for yeasts as biological agents. A transmittance measuring device is typically used to check that the turbidity of the presuspension is within the required compliance range. This presuspension is then further diluted, for example by a factor of 20 for analyzing Gram− bacteria or by a factor of 10 for analyzing Gram+ bacteria. Thus, in this example, the initial compliance of the inoculum for bacteria requires a biomass concentration (expressed as turbidity) of between 0.025 McF and 0.0315 McF for Gram− bacteria and of between 0.05 McF and 0.063 McF for Gram+ bacteria. Lower concentrations are commonly used in other protocols. This results in the concentration of biological agents in an inoculum being initially lower than the limit of detection of the transmittance measuring instruments.

However, because of the manipulations performed by the operator, there is a risk of error, or at the very least that the inoculum will not initially have the expected qualities, and therefore will not be compliant with the requirements of the analysis method. In addition, there is always the possibility of a malfunction of a part of the analysis instrument, for example a mechanical part responsible for transporting the inoculum to the wells. This unsuitability between the qualities of the initial inoculum and those expected is not immediately noticeable. Indeed, the only measurement available is the overall transmittance, and the low sensitivity thereof does not make it possible initially to stand out from the measurement background noise. A certain incubation time, typically several hours, corresponding for example to several bacterial division cycles, is necessary in order for the concentration to increase and for the transmittance to stand out from the measurement background noise.

When the inoculum does not comply, there are then two main cases:
either the initial concentration of biological agents was so low that the growth of the biological agent biomass will not be detected even after several hours of incubation (for example in a control well containing only a nutritive medium without other reagent): the analysis instrument will then report an error and the operator will have to again prepare a new inoculum;
or the initial concentration of biological agents was not compliant (too low or too high), but was sufficiently high for the growth of the biological agent biomass to be detected after several hours of incubation: no error will then be reported by the analysis instrument, but the analysis results (for example the minimum inhibitory concentration, known as “MIC”, for an antibiotic resistance test) will be erroneous.

In the first case, the loss of time caused by the late nature of the error detection can be extremely prejudicial, in particular when the results of the analysis are awaited in order to treat a patient. In the second case, the erroneous results can lead to erroneous diagnoses, and therefore to treatments which are inappropriate for a patient.

### Presentation of the Invention

The invention therefore aims to provide an analysis method and instrument for ensuring, without time loss, the reliability of the final analysis results.

To this effect, the invention provides a method for analyzing a biological sample by means of an analysis instrument, wherein, after the biological sample has been placed in an analysis receptacle in a field-of-view of a holographic imager, the receptacle comprising at least one reagent intended to interact with biological agents present in the biological sample, the method comprises the following steps carried out in a repeated manner for a plurality of measurement times during a measurement period:
acquiring an image of the biological sample,
determining, from the acquired image, a biological sample analysis criterion, and obtaining analysis results from the biological sample analysis criterion at the end of the measurement period,
   the method comprising, for a plurality of measurement times during the measurement period within a first half and a second half of the measurement period:
an acquisition of a holographic image of the biological sample by the holographic imager,
a determination, from the acquired holographic image, of a value of a distribution parameter representative of the quantitative spatial distribution of biological agents in the field-of-view, the determination of the value of the distribution parameter comprising the determination, for each of a plurality of zones of the holographic image, of the presence or absence of biological agents in said zone, the biological sample analysis criterion from which the analysis results are obtained being a value of the distribution parameter representative of the quantitative spatial distribution of biological agents, and the method also comprising, for at least one measurement time within a first half of the measurement period, an initial compliance check for the biological sample, comprising comparing the value of the distribution parameter with at least one threshold value defining a limit of a compliance range, the measuring instrument issuing a biological sample non-compliance alert if the value of the distribution parameter is outside the compliance range.

The invention is advantageously completed by the following various features taken alone or according to the various possible combinations thereof:
said threshold value is a bottom threshold value corresponding to a bottom limit of the compliance range, and if the value of the distribution parameter is lower than the bottom threshold value, the measuring instrument issues a biological sample non-compliance alert, and/or said threshold value is a top threshold value corresponding to a top limit of the compliance range, and if the value of the distribution parameter is higher than the top threshold value, the measuring instrument issues a biological sample non-compliance alert;
the biological sample initial compliance check is carried out for at least one measurement time within the first quarter of the measurement period;
the biological sample initial compliance check is carried out for at least one measurement time within the first hour of the measurement period, or within the first 30 minutes of the measurement period;
the distribution parameter is derived from a number of biological agents appearing in the holographic image;
a zone of the holographic image is between 5 and 20 times larger than a typical size of the biological agents of the biological sample;
the presence or absence of biological agents in a zone is determined by comparing a value of level of grey of the zone with a threshold, or by comparing a pattern of the zone with reference patterns of a database;
the holographic image is a hologram or an image reconstructed from a hologram;
the analysis receptacle has at least two opposite transparent faces, and the holographic image is configured so that the field-of-view extends over a field depth of at least 100 μm between the two opposite transparent faces of the analysis receptacle.

The invention also relates to an analysis instrument comprising a holographic imager with a field-of-view configured to acquire a holographic image and data processing means, the analysis instrument being configured to receive a biological sample in an analysis receptacle in the field-of-view of a holographic imager, the receptacle comprising at least one reagent intended to interact with biological agents present in the biological sample, and to implement, in accordance with the steps of the invention, for a plurality of measurement times during the measurement period within a first half and a second half of the measurement period:
an acquisition of a holographic image of the biological sample,
a determination, from the acquired holographic image, of a value of a distribution parameter representative of the quantitative spatial distribution of biological agents in the field-of-view, the determination of the value of the distribution parameter comprising determining, for each of a plurality of zones of the hologram, the presence or absence of biological agents in said zone, a biological sample analysis criterion from which the analysis results are obtained at the end of the measurement period being a value of the distribution parameter representative of the quantitative spatial distribution of biological agents, and
for at least one measurement time within a first half of a measurement period, a biological sample initial compliance check comprising comparing the value of the distribution parameter with at least one bottom threshold value, the measuring instrument issuing a biological sample non-compliance alert if the value of the distribution parameter is lower than the bottom threshold value.

### PRESENTATION OF THE FIGURES

Other features, aims and advantages of the invention will emerge from the following description, which is purely illustrative and nonlimiting, and which should be read from the viewpoint of the appended drawings in which:

FIG. 1 shows an example of an analysis card comprising a plurality of receptacles in the form of wells, which card can be used for placement of a biological sample to be analyzed, according to one possible embodiment of the invention;

FIG. 2 shows schematically an example of a holographic imager which can be used in an analysis instrument according to one possible embodiment of the invention;

FIG. 3 is a diagram illustrating steps of the method of analysis according to one possible embodiment of the invention.

### DETAILED DESCRIPTION

The method for analyzing a biological sample is carried out by means of an analysis instrument comprising a holographic imager with a field-of-view, the analysis instrument being configured to receive a biological sample in an analysis receptacle in the field-of-view of the holographic imager. The biological analysis is in this case an in vitro analysis.

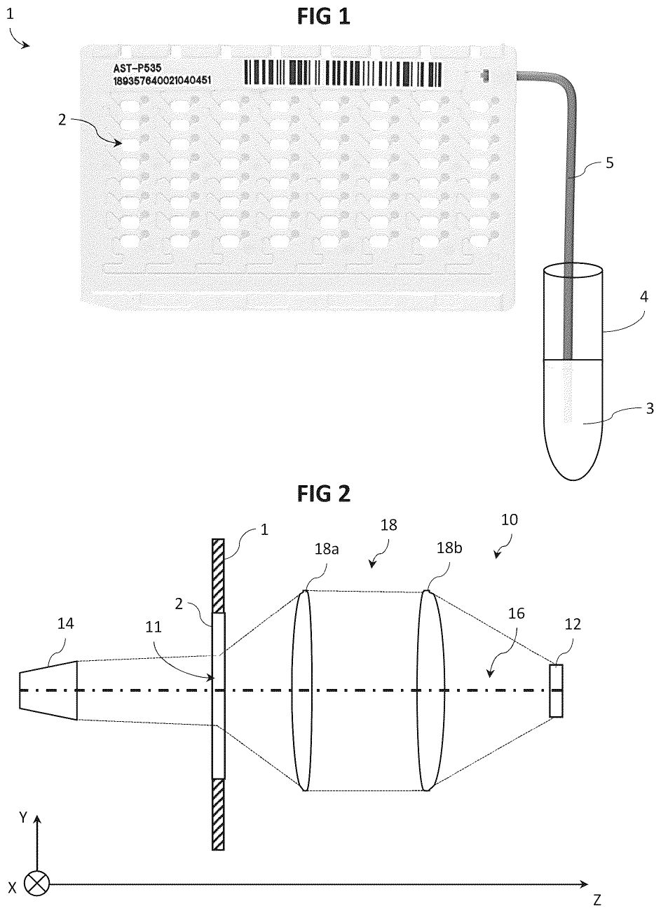

FIG. 1 shows an example of an analysis card 1 comprising a plurality of analysis receptacles 2 in the form of wells, which card can be used for placement of a biological sample to be analyzed. The analysis receptacles 2 are in this case organized in a two-dimensional network on one plane, each receptacle 2 being associated with different analysis conditions, typically by means of different reagents present in the analysis receptacles 2. For example, in the context of an antibiogram for testing antibiotic sensitivities, the reagents consist of various antibodies at various concentrations. The use of an analysis card 1 is not required, but such an analysis card makes it possible to carry out a plurality of tests in a standardized manner during one and the same analysis period.

Each analysis receptacle 2 is at least partially transparent to at least one visible or nonvisible light wavelength, and preferably is at least partially transparent for the visible spectrum. This transparency allows the analysis of the biological sample which is contained therein by optical means such as the holographic imager. Preferably, and as visible in FIG. 1, an analysis receptacle 2 has at least two opposite transparent faces, so as to have a transparent axis for light propagation. These two opposite transparent faces are for example less than 5 mm apart.

In order to allow the analysis receptacles 2 to be filled, such an analysis card 1 can for example comprise a pipe 5 intended to be immersed in a volume 3 of inoculum prepared in a tube 4. As explained above, the inoculum is prepared by an operator who introduces biological agents, for example samples from a culture in a Petri dish by means of a rod or a swab, in suspension into a saline solution, with a dilution corresponding to a predetermined turbidity range, for example between 0.5 and 0.63 McF for bacteria as biological agents or else between 1.8 and 2.2 McF for yeasts as biological agents, the range depending on the type of analysis performed and on the measuring instrument. This presuspension is then further diluted, for example by a factor 20, or even 100, for analyzing Gram− bacteria or by a factor of 10, or even 100, for analyzing Gram+ bacteria. This subsequent dilution can in particular be automated, and therefore can be performed by the measuring instrument after the placing of the tube 4 in the analysis instrument. Of course, other predetermined turbidity ranges can be used, depending on the protocols used. The desired dilution can be obtained in one step, or as in the example above, in several steps.

One end of the pipe 5 is then immersed in the volume 3 of inoculum resulting from the preparation in the tube 4, and the whole entity is introduced into the analysis instrument. Of course, all or some of these preparation steps can be automated. The inoculum travels through the pipe 5, and is then distributed between the analysis receptacles 5 by means of a fluidic circulation circuit made in the analysis card 1. This movement of the inoculum in the pipe 5 and the analysis card 1 can be caused by capillary action and/or by depressurization of the air present at the open end of the tube 4. For example with depressurization, the air present in the analysis card 1, which is atmospheric pressure, leaves the analysis card 1 via the conduit 5 through the inoculum 3 and makes way for the inoculum 3 which rises up into the analysis card 1 via the conduit 5. Conversely, it is possible to apply an air pressure which is exerted on the inoculum by means of the open end of the tube 4 in order to cause the inoculum 3 to rise up the conduit 5. The biological sample consisting of the inoculum is then in place in an analysis receptacle 2.

The analysis instrument comprises a holographic imager with a field-of-view configured to acquire a holographic image of this field-of-view. The acquisition of a holographic image allows a considerable field depth, and therefore a very good sensitivity of detection of the biological agents. During the acquisition of a holographic image, the holographic imager is placed opposite an analysis receptacle 2. By way of nonlimiting example, FIG. 2 schematically represents an online holographic imager 10 placed so that the field-of-view 11 of said holographic imager 10 is contained in the volume of biological sample contained in an analysis receptacle 2. The analysis card 1, and therefore the analysis receptacles 2 that it comprises, is placed in an object plane of the holographic imager 10. The holographic imager 10 defines an imaging axis 16, simplified here by a straight line corresponding to the optical axis but which can consist of a set of successive straight lines defining the light path, as a function of the configuration of the optical components of the holographic imager 10.

On one side of the analysis receptacle 2, in this case on the optical axis 16, is a light source 14 configured to illuminate the analysis receptacle 2 in the field-of-view of the holographic imager 10 by means of an illumination beam of sufficiently coherent light. The light source 14 can produce the illuminating light or can simply be the end of an optical fiber conveying this illuminating light, optionally provided with a diaphragm or iris. The illumination beam has the conventional characteristics for holographic imaging, without any particular additional constraints. The illumination beam can thus be monochromatic (for example with a wavelength around 640-670 nm) or can possibly be composed of several wavelengths, for example used one after the other.

On the other side of the analysis receptacle 2, in this case on the optical axis 16, is an image sensor 12, which is a digital sensor such as, for example, a CMOS or CCD sensor. The image sensor 12 is placed on an image plane of the holographic imager 10, and is configured to acquire a hologram, that is to say a spatial distribution of intensity of the interferences caused by interactions between the inoculum placed in the field-of-view 11 and the illumination beam.

The holographic imager 10 is in this case provided with a set of optical members 18 placed between the analysis receptacle 2 and the digital image sensor 12, such as for example a microscope objective 18a and a tube lens 18b in the example illustrated. An optical member such as the microscope objective 18a is however optional, the invention not being limited to holographic microscopy with lens. The arrangement described here is of course a nonlimiting example. Any holographic imager 10 can be used, with various optical members (with or without microscope objective, etc.). Thus, as long as a holographic imager 10 can acquire an image in which the interference patterns generated by the biological sample appear, this imager is suitable for carrying out the method. However, preferably, the holographic imager 10 is configured so that the field-of-view 11 extends over a field depth of at least 100 μm in the analysis receptacle 2, along the optical axis 16, and preferably extends over at least 150 μm, and more preferably over at least 250 μm. Typically, the analysis receptacle 2 comprises two opposite transparent faces organized along the optical axis 16, and the field depth extends over at least 100 μm between the two opposite transparent faces of the analysis receptacle, and preferably extends over at least 150 μm, and more preferably over at least 250 μm. The field-of-view 11 is understood to be the space in which the presence of biological agents can be determined from a hologram imaging said field-of-view 11.

The measuring instrument also comprises components which make it possible to process the data, such as a processor, a memory, a communication bus, etc. Insofar as these other components are specific only by virtue of the method that they implement and by virtue of the instructions that they contain, they are not subsequently detailed.

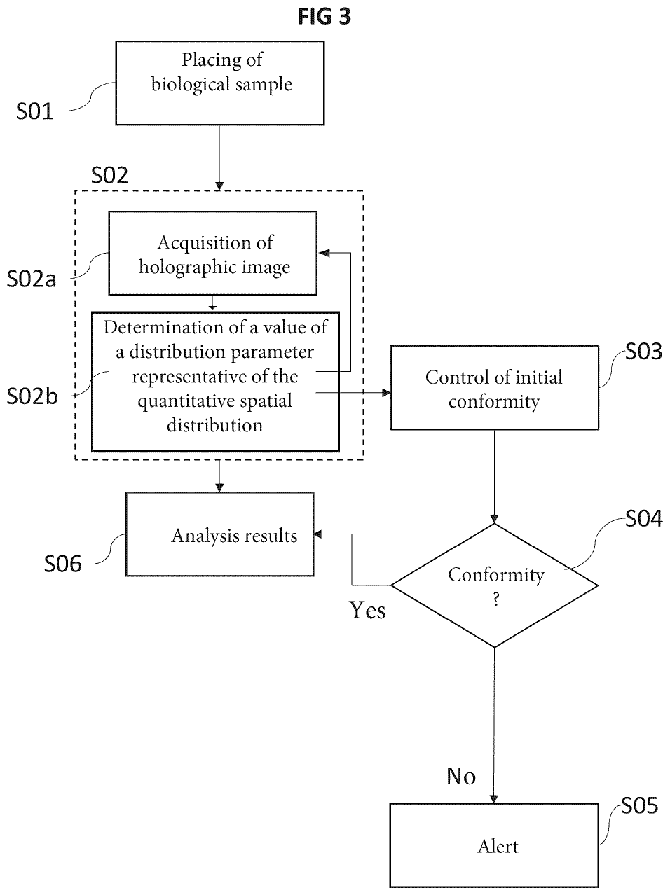

FIG. 3 is a diagram illustrating steps of the analysis method, which follow prior placement (step S01) of the biological sample in an analysis receptacle 2 in the field-of-view 11 of the holographic imager 10, detailed above. The method comprises a plurality of cycles (steps S02) consisting of steps carried out in a repeated manner for a plurality of measurement times during a measurement period:
acquiring an image of the biological sample,
determining a biological sample analysis criterion from the acquired image.

These cycles are typically repeated according to a period ranging from one minute to 30 minutes, depending on the rapidity of the analysis instrument, on the number of biological samples treated in parallel, and for example depending on the number of analysis receptacles 2 in an analysis card 1. The measurement period extends over several hours, and typically more than 10 hours, resulting in several tens of measurement times, or even several hundred measurement times. The biological sample analysis criterion can be any criterion derived from measurements on the acquired images which makes it possible to perform the analysis of the biological sample, such as for example the monitoring of a turbidity measurement by transmittance, as in the prior art.

However, the method comprises, for at least one measurement time within a first half of the measurement period:
an acquisition (step S02a) of a holographic image of the biological sample by the holographic imager 10,
a determination (step S02b) from the acquired holographic image, of a value of a distribution parameter representative of the quantitative spatial distribution of biological agents in the field-of-view 11.

It is possible for the image acquired at each measurement time during the measurement period to be a holographic image of the biological sample, and, for each acquired image, for the biological sample analysis criterion to be a value of a distribution parameter presentative of the quantitative spatial distribution of biological agents in the field-of-view 11 of the holographic imager 10. In this case, the analysis results (step S06) can be obtained from the distribution parameter values determined for each measurement time.

It is also possible for the acquisition of a holographic image of the biological sample and for the determination of the distribution parameter to be performed only for measurement times in the beginning (within a first half) of the measurement period, and not for measurement times subsequently included (within a second half of the measurement period). In this case, the values of the distribution parameter are used only for the initial compliance check, and not for obtaining the analysis results, which are therefore obtained through another biological sample analysis criterion. In this regard, it is possible, for the measurement times for which the initial compliance control is not carried out, to use a non-holographic imager to acquire the images making it possible to determine this other analysis criterion, or to use the holographic imager to acquire non-holographic images, or else to acquire holographic images without determining a distribution value, but while determining other analysis criteria from the acquired holographic images.

During the acquisition of a holographic image, the holographic imager 10 acquires a hologram, thereby having the advantage of offering a large field depth, and therefore a high sensitivity of detection of the biological agents in the biological sample. During the acquisition of a hologram, the light source 14 emits a reference illumination beam, which can result in a reference planar wave propagating in the direction Z along the imaging axis 16. The biological agents present in the field-of-view 11 inside the analysis receptacle 2, scatter the incident reference light by virtue of their diffraction properties. The wave scattered by the biological agents and the reference wave interfere on the image sensor 12 so as to form the hologram. Since a digital image sensor 12 is sensitive only to the intensity of the electromagnetic field, the hologram corresponds to the spatial distribution of intensity of the total field corresponding to the addition of the scattered wave and of the reference wave. The holographic image exploited can be the hologram or can be an image reconstructed by back propagation calculation from the hologram, using a propagation algorithm, for example based on the Rayleigh-Sommerfeld diffraction theory. Using the hologram without reconstruction makes it possible to benefit from a high sensitivity of detection, since each biological agent appears in the hologram surrounded by rings corresponding to the interference figures caused by the presence of said biological agents, accordingly facilitating the detection of the presence of these biological agents. In addition, the non-reconstruction allows calculation resource and time to be saved. However, using a reconstructed image has other advantages, such as that of making it possible to precisely localize, possibly three-dimensionally, the biological agents appearing in the reconstructed image.

The acquired holographic image contains representations of the biological agents in the field-of-view 11, spatially distributed in the holographic image. The holographic image thus makes it possible to preserve the quantitative distribution of biological agents in the field-of-view 11. Thus, if a plurality of biological agents are present in the field-of-view 11 at a plurality of positions, a plurality of representations of these biological agents will be present at a plurality of places in the holographic image. It is therefore possible to determine a distribution parameter representative of the quantitative spatial distribution of biological agents in the field-of-view 11. Thus, the distribution parameter does not account for only the overall biomass of the sample, estimated from an overall effect affecting a characteristic of the sample, as an analysis criterion such as transmittance might do, but accounts for the spatial distribution of the biological agents in the sample 1, and therefore the concentration of biological agents, by virtue of the two-dimensional information of the holographic image. The distribution parameter is thus constructed on the basis of taking into account this quantitative spatial distribution in the holographic image, which is a reflection of the quantitative spatial distribution in the sample.

This distribution parameter is for example a number of biological agents in the field-of-view 11 and appearing in the holographic image, or for example a proportion of the area of the holographic image taken up by biological agents. It is for example possible to count the number of biological agents in the holographic image. When the holographic image is a hologram, the interference patterns appear typically in the form of rings around a biological agent. A ring is a particularly easy shape to identify by means of a shape recognition algorithm, and it is therefore possible to analyze the holographic image in order to identify therein all the rings appearing therein, corresponding to as many biological agents.

In order to simplify this determination of the distribution parameter, the method can comprise determining, for each of a plurality of zones of the holographic image, typically several thousand zones, the presence or absence of biological agents in said zone. The size of the zone is chosen to be sufficiently small to allow the biological agents to be isolated without however necessarily cutting the representation of said agents. For example, the zone can be between 5 and 20 times larger than the typical size of the biological agents sought. The distribution parameter can then comprise a number of zones with the presence of biological agents for example, or can more easily correspond to a number of zones where the biological agents are absent, this being easier to demonstrate.

The determination of the presence or absence of a biological agent in a zone of the holographic image can for example be determined by comparing the average level of grey (or light intensity) in a zone with a level of grey threshold. It is also possible to perform a comparison of the pattern of the zone with a database of reference patterns corresponding to a plurality of appearances of biological agents, and to identify the reference pattern which has the greatest similarity with the zone pattern. The features associated with this reference pattern are considered to be those of the zone of pattern, which makes it possible, in addition to detecting the presence of biological agents in the zone, to deduce additional features, such as the individual growth of the biological agents, as a function of the features of the appearances about which information is provided in the database.

The cycles (steps S02) of acquisition of holographic images and of determination of distribution parameters are repeated for each analysis receptacle 2 for at least one measurement time of a plurality of measurement times during a measurement period. As mentioned above, it is possible to repeat the cycles (steps S02) of acquisition of holographic images and of determination of the value of the distribution parameter for all the measurement times. The distribution parameters thus determined can then be used to generate the analysis results. These results can for example be temporal monitoring of the change in the distribution parameters, or the identification indications which are derived therefrom. The measurement period, or incubation period, typically extends over several hours, and corresponds to the monitoring time considered to be necessary to demonstrate different changes in the biomass in the analysis receptacles 2 in order to reveal the differences in interactions between the biological agents and the reagents. However, at the beginning of this measurement period, and more precisely for at least one measurement time within the first half of the measurement period, preferably within the first quarter of the measurement period, or within the first hour of the measurement period, preferably within the first 30 minutes of the measurement period, and more preferably within the first 15 minutes of the measurement period, the method comprises an initial compliance check (step S03) with respect to the biological sample, carried out on the basis of at least one distribution parameter, in order to check that the biological sample initially has the expected qualities, and therefore complies with the requirements of the analysis method. This initial compliance check with respect to the biological sample can be carried out just once at the beginning of the measurement period, or can be carried out for a plurality of measurement times at the beginning of the measurement period: the first half of the measurement period, preferably the first quarter of the measurement period, or the first hour, preferably the first 30 minutes or more preferably the first 15 minutes of the measurement period.

The initial compliance check is based on a value of the distribution parameter determined at the beginning of the measurement period, so that any non-compliance can be detected as early as possible. The initial compliance check comprises comparing the value of the distribution parameter with at least one threshold value defining a limit of a compliance range, and the measuring instrument issues a biological sample non-compliance alert (S05) if the value of the distribution parameter is outside the compliance range.

The threshold value can be a bottom threshold value, and if the value of the distribution parameter is lower than the bottom threshold value (step S04), the measuring instrument issues a biological sample non-compliance alert (step S05). Alternatively or preferably in addition, the threshold value can be a top threshold value, higher than the bottom threshold value, and during the initial compliance check, the value of the distribution parameter is compared with this top threshold value, and if the value of the distribution parameter is higher than the top threshold value, the measuring instrument issues a biological sample non-compliance alert. The bottom threshold value corresponds to a bottom limit of a distribution parameter compliance range, while the top threshold value corresponds to a top limit of the distribution parameter compliance range.

This compliance range corresponds to the range in which the initial value of the distribution parameter must lie so that the analysis can be carried out, and in particular so as to allow a non-erroneous interpretation of the analysis results. The compliance range therefore depends on the type of analysis that is carried out and on the settings of the measuring instrument. For example, for an antibiogram of Gram+ bacteria, the compliance range can correspond to a turbidity value of between 0.05 and 0.063 McF, and can correspond to a turbidity value of between 0.025 and 0.032 McF for antibiogram of Gram− bacteria, or even less depending on the recommended dilution values. As long as the initial value of the distribution parameter is not in the compliance range (below the bottom threshold value or above the top threshold value), the biological sample does not initially have the expected qualities and is therefore non-compliant. The compliance range may be semi-open, and may for example extend from the bottom limit without top limit, or vice versa.

The biological sample non-compliance alert can take several forms. Typically, the analysis instrument comprises an electroacoustic transducer, and the issuing of the non-compliance alert comprises the issuing of a sound intended for an operator in order to warn the latter of the non-compliance. Likewise, the issuing of the non-compliance alert can comprise the issuing of a light signal intended for the operator. The analysis instrument typically comprises a human-machine interface which has a display screen, and the issuing of the non-compliance alert can comprise the displaying on the screen of a message warning an operator of the non-compliance of the inoculum, preferably while indicating the value of the distribution parameter. Other types of alerts can be envisioned, the important aspect being to warn the operator of the analysis instrument that the sample is initially non-compliant, so that this non-compliance of the sample can be remedied as fast as possible.

If the biological sample is initially compliant, that is to say when the initial value of the distribution parameter is within the compliance range, that is to say typically higher than the bottom threshold value and lower than the top threshold value, the biological sample can be analyzed with valid analysis results being obtained (step S06) at the end of the measurement period, whether these analysis results are obtained from the values of the distribution parameter or from another analysis criterion. The validity of the final analysis results is therefore dependent on the compliance of the initial sample. It is moreover possible, when the biological sample is not compliant, for the issuing of the non-compliance alert to comprise the rest of the analysis by the analysis instrument. Firstly, this is because it is needless to continue the analysis when the initial non-compliance of the biological sample shows, from the beginning of the measurement period, that the final analysis results will be unreliable, and secondly this is to prevent the determination of final analysis results which, since they are unreliable, may be dangerous when they are interpreted.

The invention is not limited to the embodiment described and represented in the appended figures. Modifications remain possible, in particular from the point of view of the constitution of the various technical features or by substitution of technical equivalents, without however departing from the field of protection of the invention.

1. A method for analyzing a biological sample by means of an analysis instrument, wherein, after the biological sample has been placed in an analysis receptacle in a field-of-view of a holographic imager, the receptacle comprising at least one reagent intended to interact with biological agents present in the biological sample, the method comprises the following steps carried out in a repeated manner for a plurality of measurement times during a measurement period:
acquiring an image of the biological sample,
determining, from the acquired image, a biological sample analysis criterion, and obtaining analysis results from the biological sample analysis criterion at the end of the measurement period,
wherein, the method comprises, for a plurality of measurement times during the measurement period within a first half and a second half of the measurement period:
an acquisition of a holographic image of the biological sample by the holographic imager,
a determination, from the acquired holographic image, of a value of a distribution parameter representative of the quantitative spatial distribution of biological agents in the field-of-view, the determination of the value of the distribution parameter comprising the determination, for each of a plurality of zones of the holographic image, of the presence or absence of biological agents in the zone, the biological sample analysis criterion from which the analysis results are obtained being a value of the distribution parameter representative of the quantitative spatial distribution of biological agents, and
the method also comprising, for at least one measurement time within a first half of the measurement period, an initial compliance check for the biological sample, comprising comparing the value of the distribution parameter with at least one threshold value defining a limit of a compliance range, the measuring instrument issuing a biological sample non-compliance alert if the value of the distribution parameter is outside the compliance range.

2. The analysis method as claimed in claim 1, wherein the threshold value is a bottom threshold value corresponding to a bottom limit of the compliance range, and if the value of the distribution parameter is lower than the bottom threshold value, the measuring instrument issues a biological sample non-compliance alert) and/or the threshold value is a top threshold value corresponding to a top limit of the compliance range, and if the value of the distribution parameter is higher than the top threshold value, the measuring instrument issues a biological sample non-compliance alert.

3. The analysis method as claimed in claim 1, wherein the biological sample initial compliance check is carried out for at least one measurement time within the first quarter of the measurement period.

4. The analysis method as claimed in claim 1, wherein the biological sample initial compliance check is carried out for at least one measurement time within the first hour of the measurement period, or within the first 30 minutes of the measurement period.

5. The analysis method as claimed in claim 1, wherein the distribution parameter is derived from a number of biological agents appearing in the holographic image.

6. The analysis method as claimed in claim 1, wherein a zone of the holographic image is between 5 and 20 times larger than a typical size of the biological agents of the biological sample.

7. The analysis method as claimed in claim 1, wherein the presence or absence of biological agents in a zone is determined by comparing a value of level of grey of the zone with a threshold, or by comparing a pattern of the zone with reference patterns of a database.

8. The analysis method as claimed in claim 1, wherein the holographic image is a hologram or an image reconstructed from a hologram.

9. The analysis method as claimed in claim 1, wherein the analysis receptacle has at least two opposite transparent faces, and the holographic imager is configured so that the field-of-view extends over a field depth of at least 100 μm between the two opposite transparent faces of the analysis receptacle.

10. An analysis instrument comprising a holographic imager with a field-of-view configured to acquire a holographic image and data processing means, the analysis instrument being configured to receive a biological sample in an analysis receptacle in the field-of-view of a holographic imager, the receptacle comprising at least one reagent intended to interact with biological agents present in the biological sample, and to implement, in accordance with claim 1, for a plurality of measurement times during the measurement period within a first half and a second half of the measurement period:
an acquisition of a holographic image of the biological sample,
a determination, from the acquired holographic image, of a value of a distribution parameter representative of the quantitative spatial distribution of biological agents in the field-of-view, the determination of the value of the distribution parameter comprising determining, for each of a plurality of zones of the hologram, the presence or absence of biological agents in the zone, a biological sample analysis criterion from which the analysis results are obtained at the end of the measurement period being a value of the distribution parameter representative of the quantitative spatial distribution of biological agents, and
for at least one measurement time within a first half of a measurement period, a biological sample initial compliance check comprising comparing the value of the distribution parameter with at least one threshold value defining a limit of a compliance range, the measuring instrument issuing a biological sample non-compliance alert if the value of the distribution parameter is outside the compliance range.
